(19) United States
(12) Patent Application Publication
(10) Pub. No.: US 2025/0197519 A1
(43) Pub. Date: Jun. 19, 2025

(54) ANTI-CD20 ANTIBODIES AND THERAPEUTIC USES THEREOF

(71) Applicant: Xencor, Inc., Pasadena, CA (US)

(72) Inventors: Matthew BERNETT, Pasadena, CA (US); Gregory MOORE, Pasadena, CA (US); John R. DESJARLAIS, Pasadena, CA (US)

(73) Assignee: Xencor, Inc., Pasadena, CA (US)

(21) Appl. No.: 18/033,165

(22) PCT Filed: Oct. 22, 2021
(86) PCT No.: PCT/US2021/056262

§ 371 Date: Apr. 21, 2023

Related U.S. Application Data
(60) Provisional application No. 63/104,952, filed on Oct. 23, 2020.

(51) Int. Cl. C07K 16/28; A61K 39/00; A61P 35/00

(52) U.S. Cl. CPC C07K 16/2887; A61P 35/00; A61K 2039/505; C07K 2317/24; C07K 2317/35; C07K 2317/52; C07K 2317/732; C07K 2317/92; C07K 2317/94

(57) ABSTRACT

Provided herein are novel anti-CD20 antibodies that exhibit several advantageous properties as compared to rituximab.

### CROSS-REFERENCE TO RELATED APPLICATIONS

This application is a 35 U.S.C. § 371 National Stage Application of International Application No. PCT/US2021/056262, filed on Oct. 22, 2021, which claims benefit of U.S. Provisional Application No. 63/104,952, filed on Oct. 23, 2020, the entire disclosure of which is each are hereby incorporated by reference.

### REFERENCE TO THE SEQUENCE LISTING

The present application is being filed along with a Sequence Listing in electronic format. The Sequence Listing is provided as a file entitled ZEN-001US1_SL created on Aug. 30, 2023, which is 13,315 bytes in size. The information in the electronic format of the sequence listing is incorporated herein by reference in its entirety.

### BACKGROUND

Antibody-based therapeutics have been used successfully to treat a variety of diseases.

### SUMMARY

Provided herein are novel monospecific and bivalent anti-CD20 antibodies that exhibit several advantageous properties as compared to rituximab. Such advantageous properties include, for example, enhanced potency of binding to CD20 expressing target cells, increased ADCC potency and prolonged serum half-life. Moreover, the anti-CD20 antibodies provided herein exhibit enhanced expression yields and greater thermostability as compared to rituximab. Also provided herein are methods of making the subject anti-CD20 antibodies and methods of treating various diseases, disorders and conditions using such subject antibodies.

In a first aspect, provided herein is an isolated anti-CD20 antibody that includes two identical heavy chain polypeptides and two identical light chain polypeptides. Each of the heavy chain polypeptides includes: i) a heavy chain variable (VH) domain that includes a vhCDR1 that includes the amino acid sequence of SEQ ID NO:3, a vhCDR2 that includes the amino acid sequence of SEQ ID NO:4, and a vhCDR3 that includes the amino acid sequence of SEQ ID NO:5; and ii) a heavy chain constant domain that includes a variant Fc domain having amino acid substitutions 239D, 332E, 428L and 434S, wherein the numbering is according to EU numbering. Each of the light chain polypeptides includes: i) a light chain variable (VL) domain that includes a vlCDR1 that includes the amino acid sequence of SEQ ID NO:9, a vlCDR2 that includes the amino acid sequence of SEQ ID NO:10, and a vlCDR3 that includes the amino acid sequence of SEQ ID NO: 11, and ii) a light chain constant domain. In this anti-CD20 antibody, the VH and VL form a CD20 binding domain; and the anti-CD20 antibody is monospecific and bivalent.

In some embodiments, the VH domain comprises the amino acid sequence of SEQ ID NO:2 and the VL domain includes the amino acid sequence of SEQ ID NO:8. In some embodiments, the heavy chain constant domain includes a human IgG1 CH1, a human IgG1 hinge region and the variant Fc domain is a variant human IgG2 Fc domain. In certain embodiments, the heavy chain constant domain includes the amino acid sequence of SEQ ID NO:6.

In another aspect, provided herein is an isolated anti-CD20 antibody that includes: a) two identical heavy chain polypeptides, wherein each heavy chain polypeptide includes the amino acid sequence of SEQ ID NO: 1; and b) two identical light chain polypeptides, wherein each light chain polypeptide includes the amino acid sequence of SEQ ID NO: 7, wherein the anti-CD20 antibody is monospecific and bivalent.

In another aspect, provided herein is a polynucleotide that includes a first polynucleotide encoding the heavy chain polypeptide of any one of the anti-CD20 antibodies described herein, and/or a second polynucleotide encoding the light chain polypeptide of any one of the anti-CD20 antibodies described herein.

In one aspect, provided herein is an expression vector that includes: a) a first polynucleotide encoding the heavy chain polypeptide of any one of the anti-CD20 antibodies described herein, and/or b) a second polynucleotide encoding the light chain polypeptide of any one of the anti-CD20 antibodies described herein.

In another aspect, provided herein is an expression vector system that includes: a) a first expression vector that includes a first polynucleotide encoding the heavy chain polypeptide of the anti-CD20 antibodies described herein, and b) a second expression vector comprising a second polynucleotide encoding the light chain polypeptide of the anti-CD20 antibodies described herein.

In one aspect, provided herein is a host cell that includes any of the polynucleotides, expression vectors, or expression vector systems described herein.

In another aspect, provided herein is a method of producing an anti-CD20 antibody that includes culturing a host cell provided described herein under conditions such that the anti-CD20 antibody is expressed, and recovering the anti-CD-20 antibody.

In an aspect, provided herein is a pharmaceutical composition that includes any of the anti-CD20 antibodies described herein.

In another aspect, provided herein is a method of treating an autoimmune disease in a subject in need thereof that includes administering to the subject an anti-CD-20 antibody as described herein. In some embodiments, the autoimmune disease is associated with a gastrointestinal, neurologic, musculoskeletal disorder. In some embodiments, the autoimmune disease is selected from rheumatoid arthritis, psoriasis, multiple sclerosis, immune thrombocytopenic purpura, myasthenia gravis, neuromyelitis optica, IgG4-related diseases, systemic Lupus Erythematosus, lupus nephritis, giant cell arteritis, takayasu disease, cold agglutinin disease, warm autoimmune hemolytic anemia, and anti-neutrophil cytoplasmic antibody (ANCA) associated vasculitides including for example, tranulomatosis with polyangiitis (GPA) (Wegener's Granulomatosis) or Microscopic Polyangiitis (MPA).

In yet another aspect, provided herein is a method of treating cancer in a subject in need thereof that includes administering to the subject an anti-CD-20 antibody as described herein. In exemplary embodiments, the cancer is non-Hodgkin's lymphoma (NHL) or chronic lymphocytic leukemia.

### BRIEF DESCRIPTION OF THE DRAWINGS

FIG. 1 depicts the amino acid sequences for Anti-CD20-Biobetter that has humanized, affinity-optimized anti-CD20 variable region and a hybrid IgG1/G2 heavy chain constant domain with S239D/1332E substitutions to enhance ADCC and M428L/N434S to extend half-life.

FIG. 2 depicts the crystal structure of the rituximab Fab arm and positions of substitutions investigated in A) engineering Stage 1 that included full humanization and a first round of affinity engineering and B) engineering Stage 2 that included additional affinity optimization. The two stages of engineering resulted in ˜200 variable heavy region variants and ˜100 variable light region variants.

FIG. 3 depicts percentage human identity to percentage human 9-mers of humanized and affinity optimized rituximab variable region CD20_H1_L1 in comparison to approved mAbs with humanized variable regions, approved mAbs with murine variable regions, and several other well-known mAbs. The analysis shows that CD20_H1_L1 variant is at least as human as marketed mAbs.

FIG. 4 depicts the expression yield from transient transfection in HEK293E cells for bivalent mAbs having various humanized/affinity-optimized variants of the rituximab variable region in comparison to bivalent mAb having the wild-type rituximab variable region. The data show that the CD20_H1_L1 Fv variant greatly enhanced expression yield in comparison to the wild-type Fv.

FIG. 5 depicts thermal stabilities (determined by DSC) of the rituximab variable region in comparison to the CD20_H1_L1 variant. The data show that the engineering enhanced the melting temperature associated with the Fab region by 6.7° C.

FIG. 6 depicts the binding of bivalent mAb having wild-type rituximab Fv to Ramos B cell line in comparison to bivalent mAb having humanized/affinity-optimized CD20_H1_L1 Fv variant. The data show that the affinity-engineering enhanced potency of binding (EC50 of 264.2 ng/mL for WT Fv vs. EC50 of 175.1 ng/mL for CD20_H1_L1 Fv in this experiment).

FIG. 7 depicts killing of Ramos B cells by bivalent mAb having WT rituximab Fv, bivalent mAb having humanized/affinity-optimized CD20_H1_L1 variant Fv, bivalent mAb having CD20_H1_L1 variant Fv and further having Hybrid IgG1/G2+S239D/I332E, and bivalent mAb having CD20_H1_L1 variant Fv and further having Hybrid IgG1/G2+S239D/1332E+M428L/N434S. The data show that affinity-optimized CD20_H1_L1 variant Fv enhances ADCC potency in comparison to WT rituximab Fv. Addition of S239D/1332E ADCC enhancing variant in the Fc further enhances ADCC potency. Addition of the M428L/N434S half-life enhancing variant in the Fe does not impact the improved ADCC conferred by the S239D/I332E variant.

FIG. 8 depicts the change in tumor volume over time in Ramos B cell-engrafted transgenic huFcRn immunocompromised RAG mice dosed with PBS, bivalent IgG1 mAb with rituximab Fv, or Anti-CD20-Biobetter. The data show that both anti-CD20 mAbs were efficacious at 5 mg/kg dose level.

FIG. 9 depicts serum concentration of bivalent IgG1 mAb with rituximab Fv and Anti-CD20-Biobetter in Ramos B cell-engrafted transgenic huFcRn immunocomprised RAG mice on the final day of the study. The data show greatly enhanced exposure for Anti-CD20-Biobetter enabled by the M428L/N434S half-life enhancing variant.

### DETAILED DESCRIPTION

### I. Overview

Provided herein are novel anti-CD20 antibodies that exhibit several advantageous properties as compared to rituximab. Such advantageous properties include, for example, enhanced potency of binding to CD20 expressing target cells, increased ADCC potency and serum half-life. Moreover, the anti-CD20 antibodies provided herein exhibit enhanced expression yields and greater thermostability as compared to rituximab. Also provided herein are methods of making the subject anti-CD20 antibodies and therapeutic uses of such subject antibodies. Aspects of the invention are discussed in further detailed below.

### II. Definitions

In order that the application may be more completely understood, several definitions are set forth below. Such definitions are meant to encompass grammatical equivalents.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

As used herein, the term “antibody” refers to traditional immunoglobulin (Ig) antibodies unless stated specifically otherwise.

Traditional immunoglobulin (Ig) antibodies are “Y” shaped tetramers. Each tetramer is typically composed of two identical pairs of polypeptide chains, each pair having one “light chain” monomer and one “heavy chain” monomer.

An antibody heavy chain typically includes a variable heavy (VH) domain (also referred to as “a heavy chain variable domain”), which includes vhCDR1-3, and an Fc domain, which includes a CH2-CH3 monomer. In some embodiments, an antibody heavy chain includes a hinge and CH1 domain. Traditional antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, IgG3, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). In some embodiments, the Fc domain is from IgG1, IgG2, IgG3 or IgG4, with IgG1 hinge-CH2-CH3 and IgG4 hinge-CH2-CH3 finding particular use in many embodiments. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” or “constant heavy domain” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (P230 in IgG1), wherein the numbering is according to the EU index as in Kabat.

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference. Other numbering conventions are available in the art and those skilled in the art would readily be able to determine the exact numbering and placement in those other numbering convention systems based on what's described herein.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL) (also referred to as “light chain variable domain”), which includes light chain CDRs vlCDR1-3, and a constant light chain region or light chain constant region (often referred to as CL or Cκ). The antibody light chain is typically organized from N- to C-terminus: VL-CL.

By “antigen binding domain” or “ABD” herein is meant a set of six Complementary Determining Regions (CDRs) that, when present as part of antibody sequences, specifically binds a target antigen (e.g., CD20) as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDRT, vhCDR2, vhCDR3 variable heavy CDRs and vlCDR1, vlCDR2 and vlCDR3 variable light CDRs. The CDRs are present in the variable heavy domain (vhCDR1-3) and variable light domain (vlCDR1-3). The variable heavy domain and variable light domain form an Fv region.

Typically, a “full CDR set” comprises the three variable light and three variable heavy CDRs, e.g., a vlCDR1, vlCDR2, vlCDR3, vhCDRT, vhCDR2 and vhCDR3. These can be part of a larger variable light or variable heavy domain, respectfully. In addition, as more fully outlined herein, the variable heavy and variable light domains can be on separate polypeptide chains, i.e., a heavy and light chain respectively.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDRT, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 27(1):55-77 (2003):

TABLE 2

| | Kabat + Chothia | IMGT | Kabat | AbM | Chothia | Contact | Xencor |
|---|---|---|---|---|---|---|---|
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)). Those skilled in the art would readily be able to determine the exact numbering and placement of the CDRs in other numbering systems described herein and known in the art.

The CDRs contribute to the formation of the antigen-binding, or more specifically, epitope binding site of the antibody. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

The six CDRs of the subject antibodies are contributed by a variable heavy and a variable light domain. In a “Fab” format, the set of 6 CDRs are contributed by two different polypeptide sequences, the variable heavy domain (vh or VH; containing the vhCDRT, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain).

By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDRT, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the VH, CH1, VL, and CL immunoglobulin domains, generally on two different polypeptide chains (e.g., VH-CH1 on one chain and VL-CL on the other). Fab may refer to this region in isolation, or this region in the context of a bispecific antibody of the invention. In the context of a Fab, the Fab comprises an Fv region in addition to the CH1 and CL domains.

By “Fv” or “Fv fragment” or “Fv region” as used herein is meant the antibody region that comprises the VL and VH domains.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution M428L refers to a variant polypeptide, in this case an Fc variant, in which the methionine at position 272 is replaced with leucine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below.

As described below, in some embodiments the parent polypeptide, for example an Fc parent polypeptide, is a human wild type sequence, such as the heavy constant domain or Fc region from IgG1 or IgG2, although human sequences with variants can also serve as “parent polypeptides”, for example the IgG1/2 hybrid of US Publication 2006/0134105 can be included. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1 or IgG2.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). Those skilled in the art would readily be able to determine the corresponding positions in other numbering systems described herein and known in the art. The modification can be an addition, deletion, or substitution.

In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “non-naturally occurring modification” as used herein is meant an amino acid modification that is not isotypic. For example, because none of the known human IgGs comprise a serine at position 434, the substitution 434S in IgG1 or IgG2 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fe Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “wild type” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody that is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction using an assay described herein or known in the art. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope. A suitable control molecule is described herein including the Example sections and known in the art.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

### III. Anti-CD20 Antibodies

Provided herein are novel monospecific and bivalent anti-CD20 antibodies that exhibit several advantageous properties as compared to rituximab. Such advantageous properties include, for example, enhanced potency of binding to CD20 expressing target cells, increased ADCC potency and increased serum half-life. Moreover, the anti-CD20 antibodies provided herein exhibit enhanced expression yields and greater thermostability as compared to rituximab. Such antibodies are particularly useful for the treatment of certain autoimmune diseases and cancers, providing a more effective and safer therapy than rituximab.

The subject anti-CD20 antibodies bind to human CD20 (Genebank Accession Numbers: NP_068769, NP_690605 and NP_690606). CD20 is a non-glycosylated surface phosphoprotein B that is expressed at particular stages of B cell development, including from late-pro-B cell stage through memory cells, as well as on malignant B cells. CD20 is a member of the membrane spanning 4-A protein family, and includes two extracellular loops. CD20 undergoes little post-translational structural modifications and is not normally shed from the cell surface. Antibodies that target CD20 are useful for the treatment of B cell-mediated autoimmune diseases, as well as lymphomas, leukemias.

The antibodies provided are monospecific and bivalent. As used herein, “monospecific” with respect to the subject antibody, refers to an antibody that only include Fv binding domains that bind to one particular target antigen. As used herein “bivalent,” with respect to the subject antibody, refers to an antibody that includes two Fv binding domains for a particular target antigen. Thus, the antibodies described herein include two Fv binding domains that each bind to human CD20.

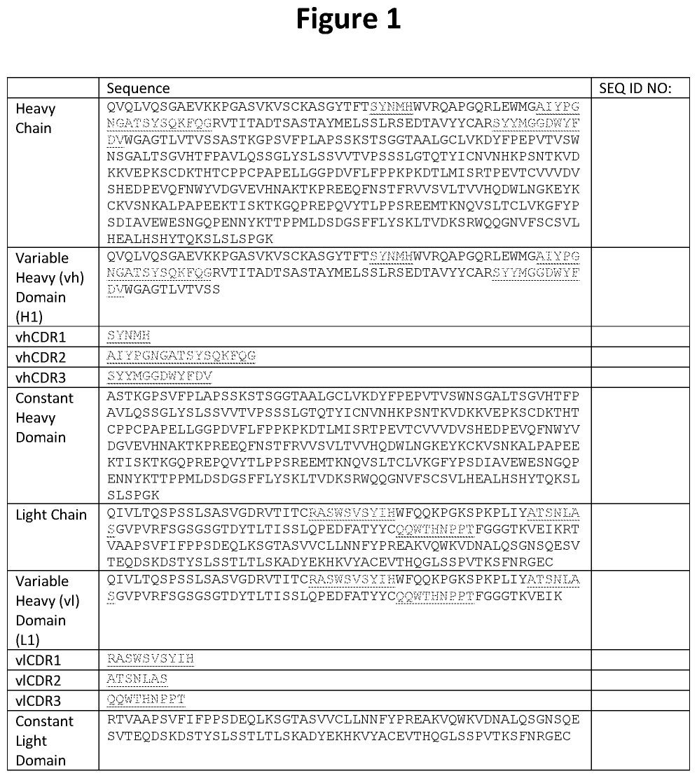

Sequences of aspects of the subject anti-CD20 antibody (Anti-CD20 Biobetter) are depicted in Table 3 below and FIG. 1.

TABLE 3

| | | SEQ |
|---|---|---|
| | | ID |
| | Sequence | NO: |
| Heavy | QVQLVQSGAEVKKPGASVKVSCKASGYTFTSYNM | 1 |
| Chain | HWVRQAPGQRLEWMGAIYPGNGATSYSQKFQGRV | |
| | TITADTSASTAYMELSSLRSEDTAVYYCARSYYM | |
| | GGDWYFDVWGAGTLVTVSSASTKGPSVFPLAPSS | |
| | KSTSGGTAALGCLVKDYFPEPVTVSWNSGALTSG | |
| | VHTFPAVLQSSGLYSLSSVVTVPSSSLGTQTYIC | |
| | NVNHKPSNTKVDKKVEPKSCDKTHTCPPCPAPEL | |
| | LGGPDVFLFPPKPKDTLMISRTPEVTCVVVDVSH | |
| | EDPEVQFNWYVDGVEVHNAKTKPREEQFNSTFRV | |
| | VSVLTVVHQDWLNGKEYKCKVSNKALPAPEEKTI | |
| | SKTKGQPREPQVYTLPPSREEMTKNQVSLTCLVK | |
| | GFYPSDIAVEWESNGQPENNYKTTPPMLDSDGSF | |
| | FLYSKLTVDKSRWQQGNVFSCSVLHEALHSHYTQ | |
| | KSLSLSPGK | |
| Variable | QVQLVQSGAEVKKPGASVKVSCKASGYTFTSYNM | 2 |
| Heavy | HWVRQAPGQRLEWMGAIYPGNGATSYSQKFQGRV | |
| (vh) | TITADTSASTAYMELSSLRSEDTAVYYCARSYYM | |
| Domain | GGDWYFDVWGAGTLVTVSS | |
| (H1.202) | | |
| vhCDR1 | SYNMH | 3 |
| vhCDR2 | AIYPGNGATSYSQKFQG | 4 |
| vhCDR3 | SYYMGGDWYFDV | 5 |
| Constant | ASTKGPSVFPLAPSSKSTSGGTAALGCLVKDYFP | 6 |
| Heavy | EPVTVSWNSGALTSGVHTFPAVLQSSGLYSLSSV | |
| Domain | VTVPSSSLGTQTYICNVNHKPSNTKVDKKVEPKS | |
| | CDKTHTCPPCPAPELLGGPDVFLFPPKPKDTLMI | |
| | SRTPEVTCVVVDVSHEDPEVQFNWYVDGVEVHNA | |
| | KTKPREEQFNSTFRVVSVLTVVHQDWLNGKEYKC | |
| | KVSNKALPAPEEKTISKTKGQPREPQVYTLPPSR | |
| | EEMTKNQVSLTCLVKGFYPSDIAVEWESNGQPEN | |
| | NYKTTPPMLDSDGSFFLYSKLTVDKSRWQQGNVF | |
| | SCSVLHEALHSHYTQKSLSLSPGK | |
| Light | QIVLTQSPSSLSASVGDRVTITCRASWSVSYIHW | 7 |
| Chain | FQQKPGKSPKPLIYATSNLASGVPVRFSGSGSGT | |
| | DYTLTISSLQPEDFATYYCQQWTHNPPTFGGGTK | |
| | VEIKRTVAAPSVFIFPPSDEQLKSGTASVVCLLN | |
| | NFYPREAKVQWKVDNALQSGNSQESVTEQDSKDS | |
| | TYSLSSTLTLSKADYEKHKVYACEVTHQGLSSPV | |
| | TKSFNRGEC | |
| Variable | QIVLTQSPSSLSASVGDRVTITCRASWSVSYIHW | 8 |
| Light | FQQKPGKSPKPLIYATSNLASGVPVRFSGSGSGT | |
| (vl) | DYTLTISSLQPEDFATYYCQQWTHNPPTFGGGTK | |
| Domain | VEIK | |
| (L1.113) | | |
| vlCDR1 | RASWSVSYIH | 9 |
| vlCDR2 | ATSNLAS | 10 |
| vlCDR3 | QQWTHNPPT | 11 |
| Constant | RTVAAPSVFIFPPSDEQLKSGTASVVCLLNNFYP | 12 |
| Light | REAKVQWKVDNALQSGNSQESVTEQDSKDSTYSL | |
| Domain | SSTLTLSKADYEKHKVYACEVTHQGLSSPVTKSF | |
| | NRGEC | |

The anti-CD20 antibodies provided herein include two heavy chain polypeptides and two light chain polypeptides. Each of the heavy chain polypeptides include a variable heavy (VH) domain and a constant heavy (CH) domain (i.e., a CH1-hinge-CH2-CH3 monomer). The variable heavy domain includes the vhCDRT-3 of the variable heavy domain according to SEQ ID NO:2. In some embodiments, the variable heavy domain of the anti-CD20 antibody includes a vhCDRT that includes the amino acid sequence of SEQ ID NO:3, a vhCDR2 that includes the amino acid sequence of SEQ ID NO:4, and a vhCDR3 that includes the amino acid sequence of SEQ ID NO:5 (Table 3). The heavy chain polypeptide further includes an Fc domain (i.e., the CH2-CH3) that is a variant Fc domain and includes ADCC potency enhancing amino acid substitutions S239D and 1332E (EU numbering), as well as half-life enhancing amino acid substitutions M428L and N434S (EU numbering). In exemplary embodiments, the constant heavy domain includes a human IgG1 isotype CH1 and hinge and a variant Fc domain that is a variant human IgG2 isotype Fc domain.

In some embodiments, the variable heavy domain of the anti-CD20 antibody includes the amino acid sequence of SEQ ID NO:2. In some embodiments, the constant heavy domain of the anti-CD20 antibody includes the sequence of SEQ ID NO:6. In exemplary embodiments, each of the two heavy chain polypeptides of the anti-CD20 antibody has the sequence of SEQ ID NO:1.

Each of the two light chain polypeptides of the subject anti-CD20 antibody include a variable light (VL) domain and a constant light (CL) domain. The variable light domain includes the vlCDR1-3 of the variable light domain according to SEQ ID NO:8. In some embodiments, the variable light domain of the anti-CD20 antibody includes a vlCDR1 that includes the amino acid sequence of SEQ ID NO:9, a vlCDR2 that includes the amino acid sequence of SEQ ID NO:10, and a vlCDR3 that includes the amino acid sequence of SEQ ID NO:11 (Table 3). In some embodiments, the variable light domain of the anti-CD20 antibody includes the amino acid sequence of SEQ ID NO:8. In some embodiments, the constant light domain of the anti-CD20 antibody includes the amino acid sequence of SEQ ID NO:12. In exemplary embodiments, each of the two light chain polypeptides of the anti-CD20 antibody includes the amino acid sequence of SEQ ID NO:7.

In some embodiments, the monospecific and bivalent anti-CD20 antibody includes two heavy chain polypeptides and two light chain polypeptide. Each of the heavy chain polypeptides includes a VH having the vhCDRT-3 of the variable heavy domain of SEQ ID NO:2 and a constant heavy domain (i.e., CH1-hinge-CH2-CH3) that includes a variant Fc (CH2-CH3) domain having amino acid substitutions S239D, 1332E, M428L and N434S (EU numbering). With respect to the constant heavy domain, this embodiment includes a human IgG1 isotype CH1 and hinge and a variant Fc domain derived from a variant human IgG2 Fc domain. Each of the two light chain polypeptides of this embodiment includes a VL having the vlCDR1-3 of the variable light domain of SEQ ID NO:8 and a constant light domain.

In some embodiments, the monospecific and bivalent anti-CD20 antibody includes two heavy chain polypeptides and two light chain polypeptide. Each of the heavy chain polypeptides includes a VH having a vhCDR1 according to SEQ ID NO:3, a vhCDR2 according to SEQ ID NO:4, and a vhCDR3 according to SEQ ID NO:5. In this embodiment, the each of the heavy chain polypeptides also include a constant heavy domain (i.e., CH1-hinge-CH2-CH3) that includes a variant Fc (CH2-CH3) domain having amino acid substitutions S239D, 1332E, M428L and N434S (EU numbering). With respect to the constant heavy domain, this embodiment includes a human IgG1 isotype CH1 and hinge and a variant Fc domain derived from a variant human IgG2 Fc domain. Each of the two light chain polypeptides of this embodiment includes a VL having a vlCDR1 according to SEQ ID NO:9, a vlCDR2 according to SEQ ID NO:10, and a vhCDR3 according to SEQ ID NO:11, and a constant light domain.

In some embodiments, the monospecific and bivalent anti-CD20 antibody includes two heavy chain polypeptides, where each heavy chain polypeptide is according to SEQ ID NO:1; and two light chain polypeptides, where each light chain polypeptide is according to SEQ ID NO:7.

As shown in the examples provided herein (see Example 1B), the subject anti-CD20 antibodies have improved thermostability and enhanced binding potency as compared to anti-CD20 antibody rituximab. Thermostability can be measured using any suitable techniques known in the art, including Differential Scanning Calorimetry (DSC) and Differential Scanning Fluorimetry (DSF) techniques. Binding potency can be measured using any suitable assay known in the art, including cell-binding, Biacore, SPR or BLI assays.

The subject antibodies provided herein exhibit enhanced ADCC potency as compared to rituximab. Such enhanced potency of the antibody is surprisingly due in part to its Fv region, as antibodies that include the Fv (i.e. VH and VL domains) of the subject antibody exhibit enhanced ADCC potency as compared to control parent antibody rituximab (see Example 1C). The subject antibodies provided herein also include amino acid substitutions S239D and 1332E (EU numbering) in the Fc domain, which further enhance the ADCC potency of the antibody by increasing FcγR affinity. ADCC can be measured using any assay known in the art, include for example, lactate dehydrogenase (LDH) activity assay. See, e.g., Horton et al., Cancer Res 68(19):8049-8057 (2008), incorporated by reference in its entirety and in pertinent parts relating to ADCC assays.

The subject antibodies provided herein include half-life enhancing amino acid substitutions M428L and N434S (EU numbering), which increases the in vivo serum half-life of the subject antibody by enhances the affinity of the subject antibody for neonatal Fc receptor (FcRn). The M428L and N434S amino acid substitutions advantageously do not negatively impact the improved ADCC conferred by the S239D/1332E variants. In some embodiments, such increased in vivo serum half-life enable the subject anti-CD20 antibodies to have longer lasting in vivo biological activity at lower and less frequent doses in comparison to control anti-CD20 rituximab antibody (see, e.g., Example 1D). FcRn binding affinity and in vivo serum half-life can be determined by any suitable method known in the art including, for examples, those assays described in Zalevsky et al., Nat. Biotechnol. 28(2):157-159 (2010), which is incorporated by reference herein, particularly in pertinent parts relating to assays for measuring FcRn binding affinity and antibody serum half-life.

### IV. Recombinant Methods and Compositions

The subject antibodies provided herein can be produced using any suitable method, including recombinant methods. See, e.g., U.S. Pat. No. 4,816,517, which is incorporated by reference herein in pertinent parts related to methods for making recombinant antibodies. Antibodies that include the Fvs (VH and VL domains, see Table 3) described herein surprisingly exhibit enhanced expression yields as compared to wildtype control rituximab antibodies (see, e.g., Example 1B).

In another aspect, provided herein are polynucleotide compositions encoding the heavy chain polypeptides and light chain polypeptides of the anti-CD20 antibodies provided herein. Also provided herein are expression vectors that include a polynucleotide encoding a heavy chain and/or a light chain polypeptide of the subject anti-CD20 provided herein. As is known in the art, the nucleic acids encoding the anti-CD20 antibodies disclosed herein can be incorporated into expression vectors as is known in the art and used to produce the subject anti-CD20 antibodies. Generally, the nucleic acids are operably linked to any number of regulatory elements (promoters, origin of replication, selectable markers, ribosomal binding sites, inducers, etc.). The expression vectors can be extra-chromosomal or integrating vectors. In some embodiments, the expression vector includes a first polynucleotide encoding a heavy chain polypeptide and second polynucleotide encoding a light chain polypeptide of an anti-CD20 antibody provided herein. In other embodiments, provided herein are expression vector compositions that include a first expression vector that includes a first polynucleotide encoding a heavy chain polypeptide of an anti-CD20 antibody provided herein and a second expression vector that includes a second polynucleotide encoding light chain polypeptide of the anti-CD20 antibody. In some embodiments, the first and second polynucleotides are under the control of the same promoter elements. In other embodiments, expression of the first and second polynucleotides is under the control of different promoter elements.

In another aspect provided herein are host cells that include the polynucleotides and/or expression vectors described herein. Suitable host cells include, but are not limited to mammalian, bacterial, yeast, insect and/or fungal cells, with mammalian cells (e.g., CHO cells). In some embodiments, the host cell is a HEK293E cell.

The anti-CD20 antibodies provided herein are made by culturing host cells comprising the expression vector(s) as is well known in the art. Once produced, traditional antibody purification steps are done, including an ion exchange chromatography step.

### V. Antibody Compositions for In Vivo Administration

In another aspect, provided herein are pharmaceutical compositions that include the subject anti-CD20 antibodies. Typically, pharmaceutical compositions provided herein further comprises a pharmaceutically acceptable excipient. For example, pharmaceutical compositions in accordance with the present invention are prepared by mixing an antibody having the desired degree of purity with optional pharmaceutically acceptable carriers, excipients or stabilizers (Remington's Pharmaceutical Sciences 16th edition, Osol, A. Ed. [1980]), in a suitable form for storage or administration including lyophilized formulations or aqueous solutions.

### VI. Therapeutic Uses

In another aspect, provided herein are methods for the treatment of autoimmune diseases, cancers and other diseases, disorders or conditions that involve CD20 mediated signaling pathways in patients in need thereof by administering a pharmaceutical composition that includes the anti-CD20 antibody described herein. The anti-CD20 antibodies provided herein are useful, for example for the treatment of rheumatoid arthritis, psoriasis, multiple sclerosis, immune thrombocytopenic purpura, myasthenia gravis, neuromyelitis optica, IgG4-related diseases, systemic Lupus Erythematosus, lupus nephritis, giant cell arteritis, takayasu disease, cold agglutinin disease, warm autoimmune hemolytic anemia, and anti-neutrophil cytoplasmic antibody (ANCA) associated vasculitides including for example, tranulomatosis with polyangiitis (GPA) (Wegener's Granulomatosis) and Microscopic Polyangiitis (MPA), pemphigus vulgaris, antiphospholipid syndrome, blistering diseases of the skin such as pemphigus and pemphigoid, myasthenia gravis, neuromyelitis optica and the inflammatory myopathies. The anti-CD20 antibodies provided herein are also useful for treatment of non-Hodkin's lymphoma, chronic lymphocytic leumia and other types of cancers.

The antibodies provided herein administered to a subject, in accord with known methods, such as intravenous administration as a bolus, intravenous push or by intravenous infusion over a period of time. In preferred embodiments, the anti-CD20 antibody provided herein is administered by intravenous infusion.

In the methods of the invention, therapy is used to provide a positive therapeutic response with respect to a disease, disorder or condition described herein. Positive therapeutic responses in any given disease, disorder or condition can be determined by standardized response criteria specific to that disease or condition.

In addition to these positive therapeutic responses, the subject undergoing therapy may experience the beneficial effect of an improvement in the symptoms associated with the disease.

Treatment according to the present invention includes a “therapeutically effective amount” of the anti-CD20 antibody used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The efficient dosages and the dosage regimens for the anti-CD20 antibodies described herein depend on the disease or condition to be treated and may be determined by the persons skilled in the art.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are known in the art and outlined in US Publications 2015/0307629, 2014/0288275 and WO2014/145806, all of which are expressly incorporated by reference in their entirety and particularly for the techniques outlined therein.

### Example 1: Engineering and Characterization of Anti-CD20-Biobetter

1A: Humanized/Affinity Optimized Anti-CD20 Fv Highly Human Compared to a Number of Existing Approved mAbs

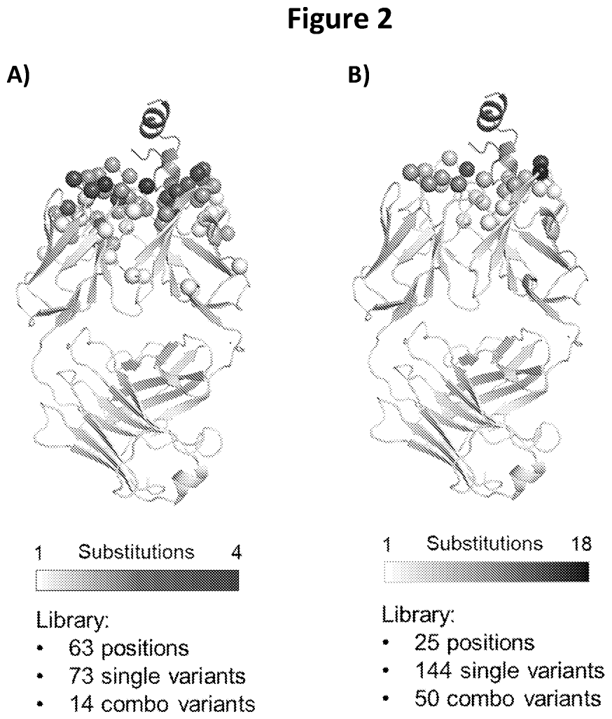

Towards generating anti-CD20 biobetter, the variable region (Fv) of rituximab were first humanized and affinity-optimized. Engineering was done in two stages with a focus on different residues (as depicted in FIG. 2). In a first stage, the Fv was humanized and affinity-engineered by introducing substitutions at 63 positions to generate 73 single variants and 14 combo variants. In a second stage, substitutions were introduced at 25 positions to generate 144 single variants and 50 combo variants. Collectively, the two stages of engineering resulted in ˜200 variable heavy region (VH) variants and ˜100 variable light region (VL) variants. The VH variants and VL variants were then combined to generate a number of humanized, affinity-optimized Fv variants. One such Fv variant is selected and referred to as [CD20]_H1_L1, sequences for which are depicted in FIG. 1.

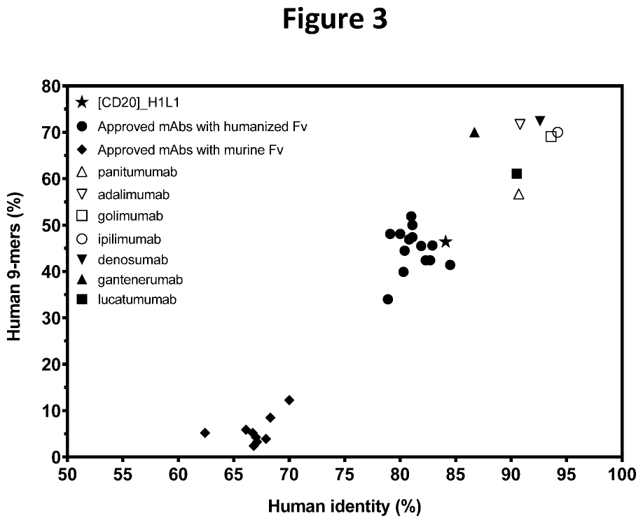

To assess the quality of the humanization effort, the percentage human 9-mers and percentage human identity of the [CD20]_H1_L1 was compared to that of approved mAbs with humanized variable regions, approved mAbs with murine variable regions, and several other well-known mAbs. The analysis as depicted in FIG. 3 showed that the [CD20]_H1_L1 variant was at least as human as marketed humanized mAbs.

### 1B: Humanized/Affinity Optimized Anti-CD20 Fv Demonstrates Enhanced Binding to CD20+ Cells and Enables Improved Production and Stability in the Context of a Bivalent IgG mAb

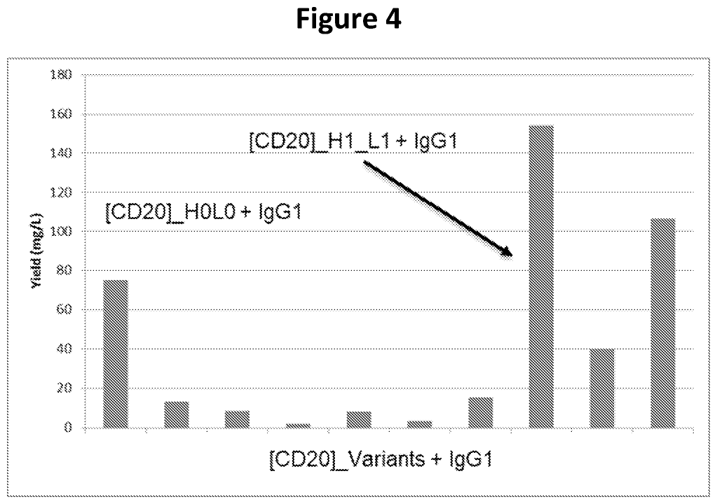

Next, the humanized, affinity-optimized anti-CD20 Fvs were formatted as bivalent mAbs on an IgG1 backbone. The antibodies were produced by transient transfection in HEK293E cells and purified. FIG. 4 depicts yield of bivalent IgG1 mAbs comprising the various humanized/affinity-optimized Fv variants including [CD20]H1_L1 in comparison to bivalent IgG1 mAb having the rituximab Fv [CD20]_H0L0. Surprisingly, as shown in FIG. 4, the [CD20]_H1_L1 Fv variant greatly enhanced expression yield in comparison to the rituximab Fv and other [CD20] Fv variants in the context of a bivalent IgG mAb.

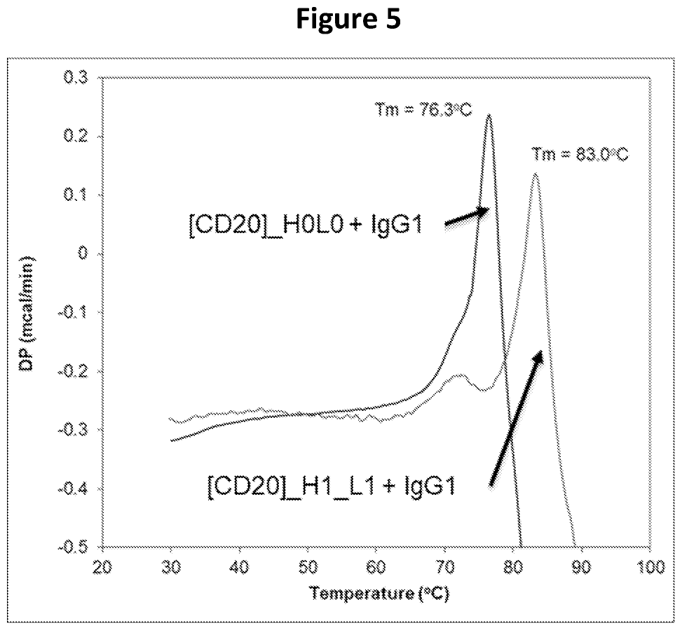

Stability of the humanized, affinity-optimized anti-CD20 Fv [CD20]_H1_L1 was investigated using Differential Scanning Calorimetry (DSC). DSC experiments were performed using a MicroCal VP-DSC. Proteins were diluted to a final concentration of 1 mg/ml in PBS and degassed before loading into the DSC. After a 15 min temperature equilibration step, 20 temperature scans were performed at a scan rate of 90° C. per hour, typically scanning within the range of 5-110° C. and with a filtering period of 8 seconds. Data was analyzed using Origin software. Stability results for bivalent IgG1 mAb having either [CD20]_H0L0 or optimized [CD20]_H1_L1 are depicted in FIG. 5. The data show that melting temperature (Tₘ) for [CD20]_H1_L1 increased by 6.7° C.

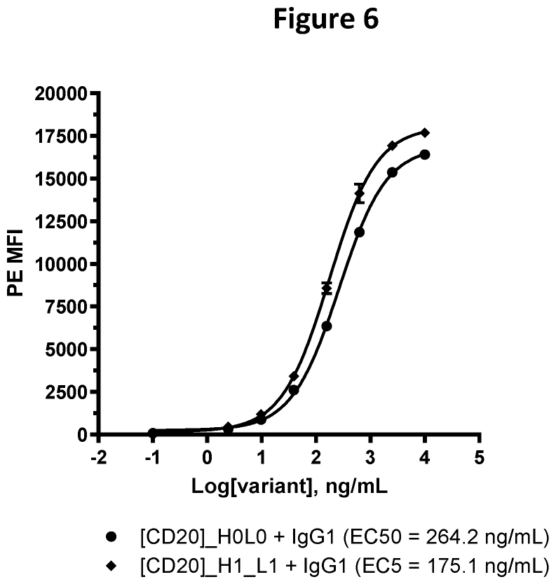

Next, binding of the improved CD20 mAb to CD20+ Ramos B cells was investigated. 1.5×10⁵ Ramos B cells were incubated with indicated concentrations of bivalent IgG1 mAb having either [CD20]H0L0 or optimized [CD20]_H1_L1 on ice for 1 hour. PE-conjugated goat anti-human IgG Fc was used as a secondary antibody to detect binding of the CD20 mAbs to the target cells. Data as depicted in FIG. 6 show that in fact the affinity-engineering enhanced potency of binding to target cells (EC50 of 264.2 ng/mL for rituximab Fv vs. EC50 of 175.1 ng/mL for [CD20]H1_L1 Fv in this experiment).

Collectively, the data show that the affinity-optimized [CD20]H1_L1 not only enables enhanced binding to target cells but also enables improved production and enhanced stability in the context of a bivalent IgG mAb.

### 1C: Affinity-Optimized CD20 Fv Enables Enhanced ADCC on CD20+ Cells, and Fc Engineering Enables Further Enhancement

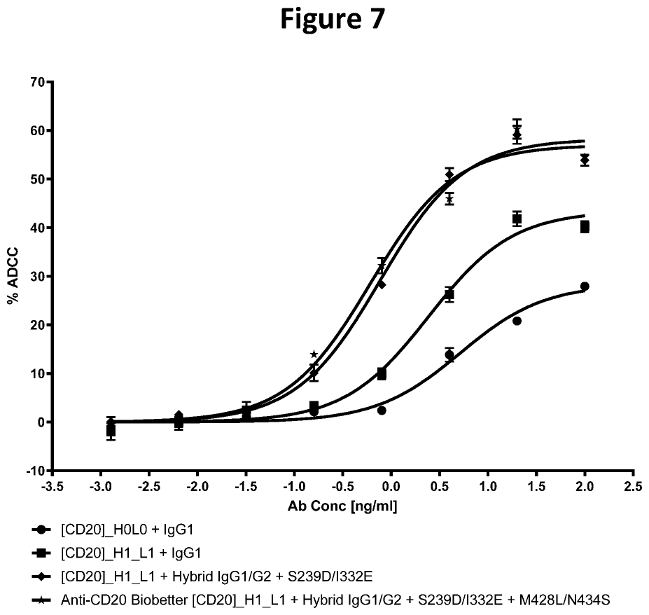

Next, the mAbs having affinity-optimized [CD20]_H1_L1 were further engineered with Fc variants to enhance ADCC (S239D/I332E) and/or enhance serum half-life (M428L/N434S) in the context of a Hybrid IgG1/G2 constant heavy domain. The additional mAbs were characterized in vitro for antibody-dependent cellular cytotoxicity (ADCC). ADCC was determined by measuring lactate dehydrogenase levels. 10,000 Ramos B cells were incubated with 500,000 human PBMCs and indicated concentrations of the indicated test articles. Data depicted in FIG. 7 show that affinity-optimized [CD20]_H1_L1 variant Fv unexpectedly enhances ADCC potency in comparison to rituximab Fv. Additional engineering of S239D/I332E in the Fc region synergistically enhances ADCC potency. Notably, addition of the M428L/N434S half-life enhancing variant in the Fc does not negatively impact the improved ADCC conferred by the S239D/1332E variant.

1D: Anti-CD20-Biobetter is Efficacious in a Mouse Anti-Tumor Model and Demonstrates Enhanced Exposure in a huFcRn Background

A monoclonal antibody with [CD20]_H1_L1 Fv region with S239D/1332E and M428L/N434S substitutions in a hybrid IgG1/G2 constant region is referred to as Anti-CD20-Biobetter (sequences shown in FIG. 1). To investigate anti-tumor activity of Anti-CD20-Biobetter, immunocompromised RAG mice transgenic for huFcRn were used. Mice were subcutaneously injected with 2.5×10⁶ Ramos lymphoma cells on Day 0. On Day 24, mice with tumors in the 100-800 mm³ range were injected intraperitoneally with 5 mg/kg of the anti-CD20 mAbs twice per week for 3 weeks. Tumors were measured by calipers two to three times per week, and blood were collected on the final day of the study.

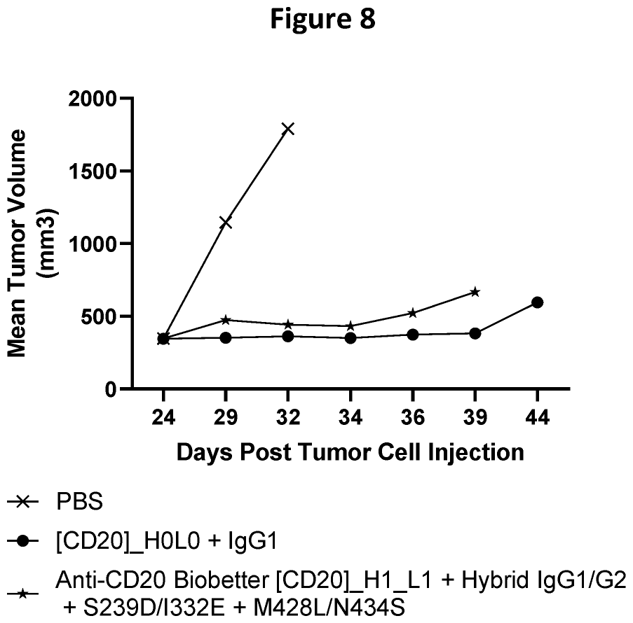

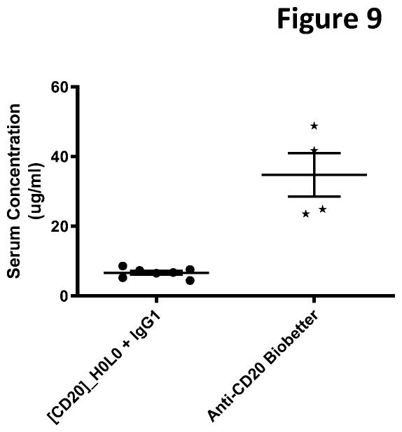

The data as depicted in FIG. 8 show that both [CD20]H0L0_IgG1 and Anti-CD20-Biobetter effectively controlled tumor growth. Notably, the data depicted in FIG. 9 show greatly enhanced exposure for Anti-CD20-Biobetter in comparison to [CD20]_H0L0_IgG1 (as indicated by serum concentration of test article in mice on the final day of the study). This suggests that at lower and less frequent dose, enhanced circulating duration of Anti-CD20-Biobetter enables longer lasting anti-tumor activity in comparison to rituximab.

1. An isolated anti-CD20 antibody comprising:
a) two identical heavy chain polypeptides, wherein each heavy chain polypeptide comprises:
i. a heavy chain variable (VH) domain comprising a vhCDR1 comprising the amino acid sequence of SEQ ID NO:3, a vhCDR2 comprising the amino acid sequence of SEQ ID NO:4, and a vhCDR3 comprising the amino acid sequence of SEQ ID NO:5,
ii. a heavy chain constant domain comprising a variant Fc domain having amino acid substitutions 239D, 332E, 428L and 434S, wherein the numbering is according to EU numbering; and
b) two identical light chain polypeptides, wherein each light chain polypeptide comprises:
i. a light chain variable (VL) domain comprising a vlCDR1 comprising the amino acid sequence of SEQ ID NO:9, a vlCDR2 comprising the amino acid sequence of SEQ ID NO: 10, and a vlCDR3 comprising the amino acid sequence of SEQ ID NO: 11, and
ii. a light chain constant domain,
wherein the VH and VL form a CD20 binding domain; and
wherein the anti-CD20 antibody is monospecific and bivalent.

2. The isolated anti-CD20 antibody of claim 1, wherein the VH domain comprises the amino acid sequence of SEQ ID NO:2 and the VL domain comprises the amino acid sequence of SEQ ID NO:8.

3. The isolated anti-CD20 antibody of claim 1, wherein the heavy chain constant domain comprises a human IgG1 CH1, a human IgG1 hinge region and wherein the variant Fc domain is a variant human IgG2 Fc domain.

4. The isolated anti-CD20 antibody of claim 3, wherein the heavy chain constant domain comprises the amino acid sequence of SEQ ID NO:6.

5. A polynucleotide comprising:
a) a first polynucleotide encoding the heavy chain polypeptide of claim 1, and
b) a second polynucleotide encoding the light chain polypeptide of claim 1.

6. An expression vector comprising:
a) a first polynucleotide encoding the heavy chain polypeptide of claim 1, and
b) a second polynucleotide encoding the light chain polypeptide of claim 1.

7. An expression vector system comprising:
a) a first expression vector comprising a first polynucleotide encoding the heavy chain polypeptide of claim 1, and
b) a second expression vector comprising a second polynucleotide encoding the light chain polypeptide of claim 1.

8. A host cell comprising the polynucleotide of claim 5.

9. A method of producing an anti-CD20 antibody comprising culturing the host cell of claim 8 under conditions such that the anti-CD20 antibody is expressed, and recovering the anti-CD-20 antibody.

10. An isolated anti-CD20 antibody comprising:
a) two identical heavy chain polypeptides, wherein each heavy chain polypeptide comprises the amino acid sequence of SEQ ID NO: 1; and
b) two identical light chain polypeptides, wherein each light chain polypeptide comprises amino acid sequence of SEQ ID NO: 7,
wherein the anti-CD20 antibody is monospecific and bivalent.

11. A polynucleotide comprising:
a) a first polynucleotide encoding a heavy chain polypeptide having the amino acid sequence of SEQ ID NO: 1; and
b) a second polynucleotide encoding a light chain polypeptide having the amino acid sequence of SEQ ID NO: 7.

12. An expression vector comprising:
a) a first polynucleotide encoding a heavy chain polypeptide having the amino acid sequence of SEQ ID NO: 1; and
b) a second polynucleotide encoding a light chain polypeptide having the amino acid sequence of SEQ ID NO: 7.

13. An expression vector system comprising:
a) a first expression vector comprising a first polynucleotide encoding a heavy chain polypeptide having the amino acid sequence of SEQ ID NO: 1; and
b) a second expression vector comprising a second polynucleotide encoding a light chain polypeptide having the amino acid sequence of SEQ ID NO: 7.

14. A host cell comprising the polynucleotide of claim 11.

15. A method of producing an anti-CD20 antibody comprising culturing the host cell of claim 14 under conditions such that the anti-CD20 antibody is expressed, and recovering the anti-CD-20 antibody.

16. A pharmaceutical composition comprising the anti-CD20 antibody of claim 1.

17. A method of treating an autoimmune disease in a subject in need thereof comprising administering to the subject the isolated antibody of claim 1.

18. The method of claim 17, wherein the autoimmune disease is associated with a gastrointestinal, neurologic, or musculoskeletal disorder.

19. The method of claim 17, wherein the autoimmune disease is selected from rheumatoid arthritis, psoriasis, multiple sclerosis, immune thrombocytopenic purpura, myasthenia gravis, neuromyelitis optica, IgG4-related diseases, systemic Lupus Erythematosus, lupus nephritis, giant cell arteritis, takayasu disease, cold agglutinin disease, warm autoimmune hemolytic anemia, and anti-neutrophil cytoplasmic antibody (ANCA) associated vasculitides, tranulomatosis with polyangiitis (GPA) (Wegener's Granulomatosis), and Microscopic Polyangiitis (MPA).

20. A method of treating cancer in a subject in need thereof comprising administering to the subject the isolated antibody of claim 1.

21. The method of claim 20, wherein the cancer is non-Hodgkin's lymphoma (NHL) or chronic lymphocytic leukemia.


SEQUENCE LISTING (12 sequences)

SEQ ID NO: 1 - protein, 451 aa, Unknown
  heavy chain (HC)
QVQLVQSGAEVKKPGASVKVSCKASGYTFTSYNMHWVRQAPGQRLEWMGAIYPGNGATSYSQKFQGRVTITADTSASTAYMELSSLRSEDTAVYYCARSYYMGGDWYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 2 - protein, 121 aa, Unknown
  Variable Heavy (vh) Domain (H1)
QVQLVQSGAEVKKPGASVKVSCKASGYTFTSYNMHWVRQAPGQRLEWMGAIYPGNGATSYSQKFQGRVTITADTSASTAYMELSSLRSEDTAVYYCARSYYMGGDWYFDVWGAGTLVTVSS

SEQ ID NO: 3 - protein, 5 aa, Unknown
  vhCDR1
SYNMH

SEQ ID NO: 4 - protein, 17 aa, Unknown
  vhCDR2
AIYPGNGATSYSQKFQG

SEQ ID NO: 5 - protein, 12 aa, Unknown
  vhCDR3
SYYMGGDWYFDV

SEQ ID NO: 6 - protein, 330 aa, Unknown
  constant heavy domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 7 - protein, 213 aa, Unknown
  Light Chain
QIVLTQSPSSLSASVGDRVTITCRASWSVSYIHWFQQKPGKSPKPLIYATSNLASGVPVRFSGSGSGTDYTLTISSLQPEDFATYYCQQWTHNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 106 aa, Unknown
  Variable Heavy (vl) Domain
QIVLTQSPSSLSASVGDRVTITCRASWSVSYIHWFQQKPGKSPKPLIYATSNLASGVPVRFSGSGSGTDYTLTISSLQPEDFATYYCQQWTHNPPTFGGGTKVEIK

SEQ ID NO: 9 - protein, 10 aa, Unknown
  vlCDR1
RASWSVSYIH

SEQ ID NO: 10 - protein, 7 aa, Unknown
  vlCDR2
ATSNLAS

SEQ ID NO: 11 - protein, 9 aa, Unknown
  vlCDR3
QQWTHNPPT

SEQ ID NO: 12 - protein, 107 aa, Unknown [sequence omitted]